(19) United States
(12) Patent Application Publication
(10) Pub. No.: US 2022/0340503 A1
(43) Pub. Date: Oct. 27, 2022

(54) BRANCHED ALKANES AND PROCESS FOR PREPARING SAME

(71) Applicant: L'OREAL, Paris (FR)

(72) Inventors: Serge RATTON, Saint Germain En Laye (FR); MArc LEMAIRE, Villeurbanne (FR); Luc MATHIS, Le Kremlin Bicetre (FR); Estelle METAY, Villeurbanne (FR)

(21) Appl. No.: 17/635,427

(22) PCT Filed: Aug. 17, 2020
(86) PCT No.: PCT/EP2020/072987

§ 371 Date: Feb. 15, 2022

(30) Foreign Application Priority Data
Aug. 16, 2019 (FR) FR1909242
Mar. 27, 2020 (FR) FR2003014

(51) Int. Cl. C07C 5/03; C07C 2/30

(52) U.S. Cl. CPC C07C 5/03; C07C 2/30

(57) ABSTRACT

The present application relates to branched alkanes comprising n carbon atoms, n being an integer between 9 and 50, to the process for preparing same and to uses thereof. The present application also relates to the olefins for obtaining these branched alkanes.

The present invention relates to branched alkanes or a mixture of branched alkane isomers comprising n carbon atoms, n representing an integer between 9 and 50. The present application also relates to branched alkanes or a mixture of branched alkane isomers comprising n carbon atoms, n representing 16, 24 or 32, said alkane or mixture of alkanes being free from branched alkane comprising n−4 or n+4 carbon atoms. Finally, the present application relates to branched olefins making it possible by hydrogenation to obtain the alkanes of the invention.

Branched alkanes comprising a large number of carbon atoms, in particular 9 carbon atoms or more, preferably 16 carbon atoms or more, have varied applications. They can in particular be used as ingredients in cosmetic formulations, in agrochemical formulations, as plasticising additives, lubricants, etc., in formulations belonging to various other fields of application.

However, these compounds generally come from fossil resources, in particular petroleum. In addition to having a negative impact on the environment, the use of fossil resources, and in particular petroleum, result in alkanes having impurities of the aromatic compound type. In addition, to obtain higher alkanes, in particular with a number of carbon atoms of at least 16, it is necessary in particular to pass through oligomerisation reactions, these reactions lead to mixtures of olefins and then to mixtures of alkanes (after hydrogenation of the olefins) comprising n carbon atoms which have impurities of n−4 and n+4 carbon atoms. Such impurities are not desired since they are, in the case of n−4, too volatile and, in the case of n+4, too viscous, compared with the properties sought.

It is therefore necessary to be able to provide higher branched alkanes preferably having in particular a lower level of impurities.

One objective of the present invention is consequently to provide higher branched alkanes, in particular comprising n carbon atoms, n representing an integer between 9 and 50, preferably comprising 16, 24, 32, 40 or 48 carbon atoms.

Another objective of the present invention is to provide such alkanes, in particular comprising 16, 24, 32, 40 or 48 carbon atoms, having a lower level of impurities.

Another objective of the present invention is also to provide a method for preparing such alkanes.

Yet other objectives will emerge from the reading of the following description of the invention.

The present application relates to a branched alkane comprising n carbon atoms, n being an integer between 9 and 50, preferably n is equal to 16, 24, 32, 40 or 48, preferably the alkanes for which n represents 16, 24, 32, 40 or 48 are free from branched alkane comprising n−4 or n+4 carbon atoms.

Preferably n is equal to 12.

In the context of the present invention, the fact that the branched alkane is free from alkane comprising n−4 or n+4 carbon atoms means that the alkane does not include, as impurities, alkanes comprising n−4 or n+4 carbon atoms.

Preferably, the alkanes according to the invention are of the following formula (I):

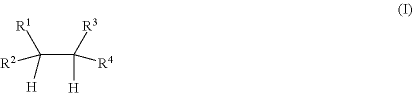

R¹, R², R³ and R⁴, identical or different, are selected from H, the alkyls, linear or branched, comprising 1 to 46 carbon atoms and the total number of carbon atoms in the R¹, R², R³ and R⁴ groups being between 7 and 48;
provided that:
at most two of the R¹, R², R³ and R⁴ groups are H;
one of the R¹, R², R³ or R⁴ groups includes or is a tert-butyl group.

Preferably, one of the R¹, R², R³ or R⁴ groups is a methyl group.

Preferably, the total number of carbon atoms in the R¹, R², R³ and R⁴ groups is equal to 10.

Preferably, the alkanes according to the invention are of the following formula (I):

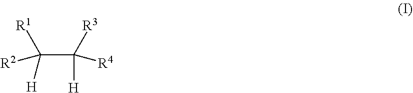

R¹, R², R³ and R⁴, identical or different, are selected from H, the alkyls, linear or branched, comprising 1 to 46 carbon atoms and the total number of carbon atoms in the R¹, R², R³ and R⁴ groups being equal to 14, 22, 30, 38 or 46; provided that:
at most two of the R¹, R², R³ and R⁴ groups are H;
one of the R¹, R², R³ or R⁴ groups includes or is a tert-butyl group.

Preferably, the alkanes according to the invention are of the following formula (I):

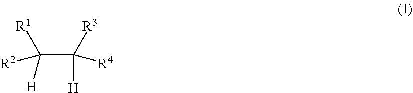

R¹, R², R³ and R⁴, identical or different, are selected from H, the alkyls, linear or branched, comprising 1 to 46 carbon atoms and the total number of carbon atoms in the R¹, R², R³ and R⁴ groups being equal to 14, 22, 30, 38 or 46; provided that:
at most two of the R¹, R², R³ and R⁴ groups are H.

Preferably, the alkanes according to the invention are of the following formula (I):

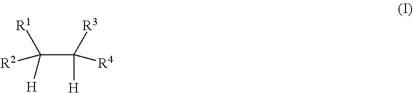

R¹, R², R³ and R⁴, identical or different, are selected from H, the alkyls, linear or branched, comprising 1 to 46 carbon atoms and the total number of carbon atoms in the R¹, R², R³ and R⁴ groups being equal to 14, 22, 30, 38 or 46; provided that:
at most two of the R¹, R², R³ and R⁴ groups are H;
when one of the R¹ or R² groups is H or when the R₁ and R₂ groups are H, then the R³ and R⁴ groups are (C₁-C₄₆) alkyl groups, and
when one of the R₃ or R₄ groups is H or when the R₃ and R₄ groups are H, then the R¹ and R² are (C₁-C₄₆)alkyl groups.

Preferably, the alkanes according to the invention are of the following formula (I):

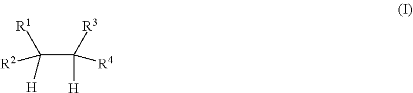

R¹, R², R³ and R⁴, identical or different, are selected from H, the alkyls, linear or branched, comprising 1 to 46 carbon atoms and the total number of carbon atoms in the R¹, R², R³ and R⁴ groups being equal to 14, 22, 30, 38 or 46; provided that:
at most two of the R¹, R², R³ and R⁴ groups are H;
when one of the R¹ or R² groups is H or when the R₁ and R₂ groups are H, when the R³ and R⁴ groups are (C₁-C₄₆)alkyl groups, and
when one of the R₃ or R₄ groups is H or when the R₃ and R₄ groups are H, then the R¹ and R² are (C₁-C₄₆)alkyl groups
   one of the R¹, R², R³ or R⁴ groups includes or is a tert-butyl group.

According to the invention, an alkyl group designates a saturated hydrocarbon aliphatic group, linear or branched, comprising, unless mentioned to the contrary, 1 to 46 carbon atoms. By way or examples, mention can be made of the methyl, ethyl, n-propyl, isopropyl, butyl, isobutyl, tert-butyl, pentyl, undecenyl, lauryl, palmyl, oleyl, linoleyl, erucyl or ricinoleyl groups.

In the aforementioned formula (I), according to one embodiment, one of the R¹, R², R³ or R⁴ groups includes a tert-butyl group. According to one embodiment, in the formula (I), one of the R¹, R², R³ or R⁴ groups is a tert-butyl group.

According to one embodiment, in the formula (I), one of the R¹, R², R³ or R⁴ groups includes a tert-butyl group and has the formula -A-C(CH₃)₃, A representing an alkylene radical comprising 1 to 6 carbon atoms.

The term “alkylene” designates according to the invention a radical comprising 1 to 6 carbon atoms, and preferably 1 to 4 carbon atoms. An alkylene radical corresponds to an alkyl radical as defined here from which a hydrogen atom has been removed.

Preferably, the alkanes according to the invention are free from aromatic compounds.

The present application also relates to mixtures of branched alkane isomers according to the invention comprising n carbon atoms, n being an integer between 9 and 50. In the context of the present invention, the mixture of isomers may be composed of various alkane isomers comprising n carbon atoms, n having a single value between 9 and 50, or a mixture of alkane isomers for which the values of n are different.

The present application also relates to mixtures of branched alkane isomers comprising n carbon atoms, n being equal to 16, 24, 32, 40 or 48, said mixture being free from branched alkane comprising n−4 or n+4 carbon atoms.

In the context of the present invention, the mixture of isomers may be composed of various alkane isomers comprising n carbon atoms, n having a single value selected from 16, 24, 32, 40 or 48. The mixture of isomers according to the invention may also be composed of various alkane isomers comprising 16 carbon atoms and/or 24 carbon atoms and/or 32 carbon atoms and/or 40 carbon atoms and/or 48 carbon atoms.

Preferably, the mixtures of branched alkane isomers according to the invention are free from aromatic compounds.

Preferably, the mixture of branched alkane isomers according to the invention comprises at least two branched alkane isomers of the following formula (I):

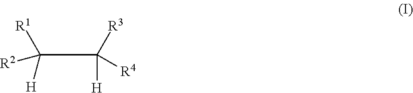

R¹, R², R³ and R⁴, identical or different, are selected from H, the alkyls, linear or branched, comprising 1 to 46 carbon atoms and the total number of carbon atoms in the R¹, R², R³ and R⁴ groups being between 7 and 48;
provided that:
at most two of the R¹, R², R³ and R⁴ groups are H;
one of the R¹, R², R³ or R⁴ groups includes or is a tert-butyl group.

Preferably, one of the R¹, R², R³ or R⁴ groups is a methyl group.

Preferably, the mixture of branched alkane isomers according to the invention comprises at least two branched alkane isomers of the following formula (I):

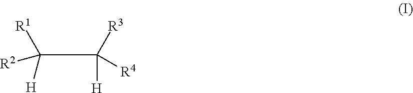

R¹, R², R³ and R⁴, identical or different, are selected from H, the alkyls, linear or branched, comprising 1 to 46 carbon atoms and the total number of carbon atoms in the R¹, R², R³ and R⁴ groups being between 7 and 48;
provided that:
at most two of the R¹, R², R³ and R⁴ groups are H;
when one of the R¹ or R² groups is H or when the R₁ and R₂ groups are H, then the R³ and R⁴ groups are (C₁-C₄₆)alkyl groups, and
when one of the R₃ or R₄ groups is H or when the R₃ and R₄ groups are H, then the R¹ and R² groups are (C₁-C₄₆)alkyl groups
one of the R¹, R², R³ or R⁴ groups includes or is a tert-butyl group.

Preferably, one of the R¹, R², R³ or R⁴ groups is a methyl group.

Preferably, the mixture of branched alkane isomers according to the invention comprises at least two branched alkane isomers of the following formula (I):

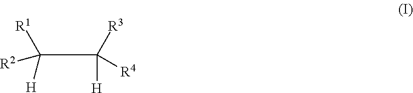

R¹, R², R³ and R⁴, identical or different, are selected from H, the alkyls, linear or branched, comprising 1 to 46 carbon atoms and the total number of carbon atoms in the R¹, R², R³ and R⁴ groups being equal to 14, 22, 30, 38 or 46; provided that:
at most two of the R¹, R², R³ and R⁴ groups are H;
one of the R¹, R², R³ or R⁴ groups includes or is a tert-butyl group.

Preferably, the mixture of branched alkane isomers according to the invention comprises at least two branched alkane isomers of the following formula (I):

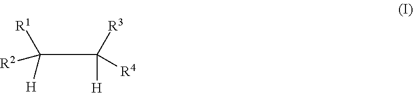

R¹, R², R³ and R⁴, identical or different, are selected from H, the alkyls, linear or branched, comprising 1 to 46 carbon atoms and the total number of carbon atoms in the R¹, R², R³ and R⁴ groups being equal to 14, 22, 30, 38 or 46; provided that:
at most two of the R¹, R², R³ and R⁴ groups are H;
when one of the R¹ or R² groups is H or when the R₁ and R₂ groups are H, then the R³ and R⁴ groups are (C₁-C₄₆)alkyl groups, and
when one of the R₃ or R₄ groups is H or when the R₃ and R₄ groups are H, then the R¹ and R² are (C₁-C₄₆)alkyl groups.

Preferably, the mixture of branched alkane isomers according to the invention comprises at least two branched alkane isomers of the following formula (I):

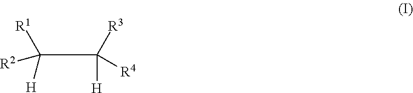

R¹, R², R³ and R⁴, identical or different, are selected from H, the alkyls, linear or branched, comprising 1 to 46 carbon atoms and the total number of carbon atoms in the R¹, R², R³ and R⁴ groups being equal to 14, 22, 30, 38 or 46; provided that:
at most two of the R¹, R², R³ and R⁴ groups are H;
when one of the R¹ or R² groups is H or when the R₁ and R₂ groups are H, then the R³ and R⁴ groups are (C₁-C₄₆)alkyl groups, and
when one of the R₃ or R₄ groups is H or when the R₃ and R₄ groups are H, then the R¹ and R² are (C₁-C₄₆)alkyl groups
one of the R¹, R², R³ or R⁴ groups includes or is a tert-butyl group.

According to one embodiment, the mixtures of the invention are such that all the compounds of formula (I) as defined above comprise n carbon atoms, n being as defined above and being identical for all the compounds of said mixture.

Preferably, said mixtures are free from aromatic compounds.

The present application also relates to a method for preparing branched alkanes or a mixture of branched alkanes according to the invention. This preparation method comprises a step of hydrogenation of branched olefins or mixture of branched olefins comprising n carbon atoms, n being defined above, preferably n being equal to 16, 24, 32, 40 or 48. Preferably, when n is equal to 16, 24, 32, 40 or 48, the branched olefins or mixture of branched olefins are free from olefins comprising n−4 or n+4 carbon atoms, and preferably free from aromatic compounds.

The hydrogenation step corresponds to putting the branched olefin or mixtures of branched olefins together with dihydrogen (H₂).

The hydrogenation step can be implemented in the presence of a hydrogenation catalyst selected from the derivatives of metals such as Pd, Pt, Ni, in solution when they are put in the form of organometallic complexes or in a form supported on solids such as silica, alumina or carbon, and preferably Raney nickel.

The hydrogenation step can be implemented without solvents or with solvent present, the solvent can in particular be selected from the alkanes that can be separated from the branched alkyls obtained as a result of the hydrogenation by techniques known to a person skilled in the art, in particular isooctane, ethers, for example diisopropylether, dibutylether, or heavy alcohols, for example alcohols comprising more than 4 carbon atoms, for example octanol, decanol, dodecanol, isododecanol. Preferably, the solvents are biosourced solvents (coming from biological resources), in particular isododecanol coming from biosourced isododecanol.

The hydrogenation step is preferably implemented at a temperature of between 50 and 150° C., for example at 80° C.

During the hydrogenation step, the hydrogen is introduced by adjusting the pressure to a constant value of between 1.013×10⁶ and 5.066×10⁶ Pa, for example 2.027×10⁶ Pa.

Preferably, the hydrogenation step has a duration of between 2 and 6 hours, for example 3 hours.

At the end of the hydrogenation step, the excess hydrogen can be eliminated by pressure reduction and the reactor is purged three times with an inert gas, preferably nitrogen.

The catalyst, if it is heterogeneous, can be recovered by filtration and can be recycled. The solvent of the reaction can be separated by distillation and can be recycled. In addition, continuous reactors can advantageously be used.

If necessary, the isomers of the branched alkanes according to the invention can be separated and purified by distillation.

The present invention also relates to a branched olefin comprising n carbon atoms, n being an integer between 9 and 50, preferably n being equal to 16, 24, 32, 40 or 48. Preferably, the branched olefin is free from aromatic compounds. Preferably, the branched olefin, when n is equal to 16, 24, 32, 40 or 48, is free from olefin comprising n−4 or n+4 carbon atoms, and preferably free from aromatic compounds.

The branched olefin according to the invention is preferably of formula (II)

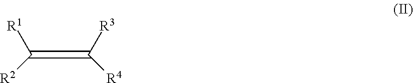

wherein R¹, R², R³ and R⁴ have the definition given for formula (I).

Preferably, in this formula (II):
at most two of the R¹, R², R³ and R⁴ groups are H,
when one of the R¹ or R² groups is H or when the R¹ and R² groups are H, then the R³ and R⁴ groups are (C₁-C₄₆)alkyl groups, and
when one of the R³ and R⁴ groups is H or when the R₃ and R₄ groups are H, then the R¹ and R² groups are (C₁-C₄₆)alkyl groups.

Preferably, the compounds of formula (II) are branched compounds comprising a total 8+4× carbon atoms with x representing 2, 4, 6, 8 or 10. The compounds of formula (II) are therefore branched compounds the main chain of which comprises 16, 24, 32, 40 or 48 carbon atoms.

The olefins of formula (II) therefore have one of the following formulae:

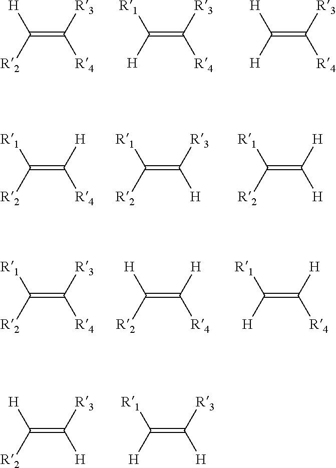

wherein R′₁, R′₂, R′₃ and R′₄ are (C₁-C₃₀)alkyl groups preferably

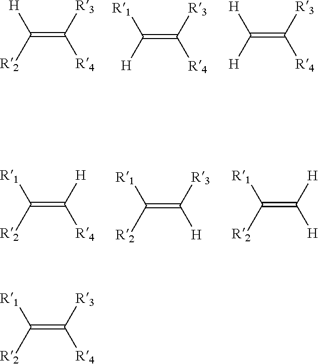

According to one embodiment, the olefins of formula (II) may comprise two hydrogen atoms, one corresponding to the R′₁ or R′₂ group and the other to the R′₃ or R′₄ group.

The mixture of branched olefin isomers according to the invention is a mixture comprising at least two olefins of formula (II). Preferably, the mixtures of the invention are such that all the compounds of formula (II) as defined above comprise n carbon atoms, n being as defined above and being identical for all the compounds of said mixture.

The present invention also relates to a mixture of branched olefin isomers comprising n carbon atoms, n being equal to 16, 24, 32, 40 or 48, the mixture of branched olefin isomers being free from olefin comprising n−4 or n+4 carbon atoms, and preferably free from aromatic compounds. Preferably, the branched olefins are of formula (II).

The present invention also relates to branched olefins comprising n carbon atoms, n representing an odd number between 9 and 49 or n represents 10, 14, 18, 22, 26, 30, 32, 34, 36, 40, 42, 44, 46, 50.

Preferably, the olefins then have the following formula (III):

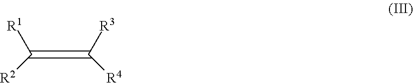

wherein R¹, R², R³ and R⁴, identical or different, are selected from H, alkyls, linear or branched, at least one of these alkyls being branched, comprising 1 to 48 carbon atoms, and the total number of carbon atoms of formula (I) is equal to n; provided that:
at least two of R¹, R², R³ and R⁴ are different from H; and
R¹ and R² cannot be simultaneously H; and
R³ and R⁴ cannot be simultaneously H.

Preferably, in this formula (III), no more than one of R¹, R², R³ and R⁴ is H.

Preferably, in this compounds of formula (III), R¹ is H or linear or branched alkyl comprising 1 to 15 carbon atoms, and R², R³ and R⁴, identical or different, are selected from the alkyls, linear or branched, comprising 1 to 15 carbon atoms.

Preferably, in the compounds of formula R¹ is H, R² is an alkyl, linear, comprising 1 to 15 carbon atoms, R³ and R⁴, identical or different, are selected from the alkyls, linear or branched, comprising 1 to 15 carbon atoms.

Preferably, the olefins then have the following formula

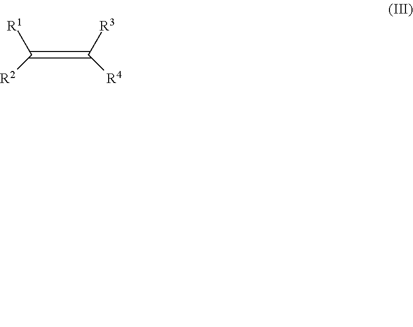

wherein R¹, R², R³ and R⁴, identical or different, are selected from H, alkyls, linear or branched, at least one of these alkyls being branched, comprising 1 to 48 carbon atoms, and the total number of carbon atoms of formula (I) is equal to n; provided that:
at least two of R¹, R², R³ and R⁴ being different from H.

Preferably, the olefins then have the following formula

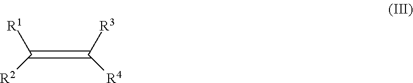

wherein R¹, R², R³ and R⁴, identical or different, are selected from H, alkyls, linear or branched, at least one of these alkyls being branched, comprising 1 to 48 carbon atoms, and the total number of carbon atoms of formula (I) is equal to n; provided that:
at least two of R¹, R², R³ and R⁴ are different from H,
one of the R¹, R², R³ or R⁴ groups includes or is a tert-butyl group.

Preferably, the olefins then have the following formula (III):

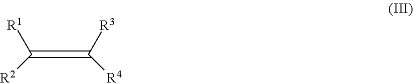

wherein R¹, R², R³ and R⁴, identical or different, are selected from H, alkyls, linear or branched, at least one of these alkyls being branched, comprising 1 to 48 carbon atoms, and the total number of carbon atoms of formula (I) is equal to n; provided that:
at least two of R¹, R², R³ and R⁴ are different from H; and
R¹ and R² cannot be simultaneously H; and R³ and R⁴ cannot be simultaneously H
one of the R¹, R², R³ or R⁴ groups includes or is a tert-butyl group.

The branched olefins or mixture of branched olefin isomers according to the invention comprising n carbon atoms, n being equal to 16, 24, 32, 40 or 48 carbon atoms, can be obtained by dimerisation of a mixture of branched olefin isomers comprising n/2 carbon atoms.

Thus the branched olefins comprising 16 carbon atoms can be obtained by dimerisation of branched olefins comprising 8 carbon atoms. The branched olefins comprising 24 carbon atoms can be obtained by dimerisation of branched olefins comprising 12 carbon atoms. The branched olefins comprising 32 carbon atoms can be obtained by dimerisation of branched olefins comprising 16 carbon atoms. The branched olefins comprising 40 carbon atoms can be obtained by dimerisation of branched olefins comprising 20 carbon atoms. The branched olefins comprising 48 carbon atoms can be obtained by dimerisation of branched olefins comprising 24 carbon atoms.

The branched olefin isomers comprising n/2 carbon atoms may have undergone a purification, in particular by distillation, before the dimerisation step.

The dimerisation step can be implemented in the presence of a catalyst selected from Brönsted acids in solution, for example H₂SO₄, H₃PO₄, HF, methanesulfonic acid, triflic acid (CF₃SO₃H); solid Brönsted acids, for example organic resins, clays, zeolites, H₃PO₄ on silica; Lewis acids, for example ZnCl₂, AlCl₃; organometallic compounds, for example Ni complexes, mixtures of complexes of Ni and Al; ionic liquids, for example [BMIm][N(CF₃SO₂)₂]/HN(CF₃SO₂)₂; clays with lamellar structures such as Montmorillonite; organic resins such as amberlysts, sulfonic resins; organometallic compounds such as for example [LNiCH₂R⁹][AlCl₄] wherein L=PR¹⁰, R⁹ represents an alkyl, linear or branched, comprising 9 carbon atoms, and R¹⁰ represents a —CH₂—R⁹ group.

The dimerisation step is preferably implemented at a temperature of between 30 and 250° C., preferably between 100 and 200° C.

Particularly advantageously, the branched olefins comprising 8, 12 and 16 carbon atoms were obtained from isobutene. Preferably, said isobutene is obtained from bioresources, in particular as described in the applications WO 2012052427,

WO 2017085167 and WO 2018206262, for example from polysaccharides (sugars, starches, celluloses, etc.).

The olefins (II) of the invention can also be obtained by codimerisation of lower olefins or by metathesis of lower olefins.

In the context of the present invention, lower olefins means olefins comprising fewer than n carbon atoms.

The codimerisation method is implemented between an olefin comprising m carbon atoms and an olefin comprising p carbon atoms, m and p being integer numbers selected so that m+p=n with n representing an integer between 9 and 50.

The lower olefins used in the codimerisation method may for example be of formula (IV) and (V):

R⁵R⁶C═CR⁷R⁸ (IV)

R⁹R¹⁰C═CR¹¹R¹² (V)

the olefin (IV) being an exo olefin (terminal double bond) or endo olefin (non-terminal double bond) comprising m carbon atoms, the olefin (V) comprising p carbon atoms, with m+p=n with n representing an integer between 9 and 50, m between 4 and 32 and p between 3 and 46,

thus, in the formulae (IV) and (V)

R⁷ and R⁸ represent H and R⁵ and R⁶, identical or different, represent an alkyl group, linear or branched, comprising in total, with the carbon atoms carrying the double bond, m carbon atoms; or

R⁵, R⁶, R⁷ and R⁸, identical or different, represent a linear or branched alkyl group comprising in total, with the carbon atoms carrying the double bond, m carbon atoms; or

R⁵ represents H and R⁶, R⁷ and R⁸, identical or different, represent a linear or branched alkyl group comprising in total, with the carbon atoms carrying the double bond, m carbon atoms; R⁹, R¹⁰, R¹¹ and R¹², identical or different, represent an alkyl group, linear or branched, comprising in total, with the carbon atoms carrying the double bond, p carbon atoms; or

R⁹, R¹¹ and R¹² represent H and R¹⁰ represents an alkyl group, linear or branched, comprising in total, with the carbon atoms carrying the double bond, p carbon atoms.

The metathesis method is implemented between an olefin comprising q carbon atoms and an olefin comprising r carbon atoms, q and r being integer numbers selected so that q+r is greater than n with n representing an integer between 9 and 50. The metathesis reaction causes the loss of carbon atoms in the final compound (loss of at least two carbon atoms), the number of carbon atoms lost being dependent on the olefins involved and in particular the nature of the substituents of the two carbon atoms of the double bond.

The lower olefins used in the metathesis method may for example be of formula (VI) and (VII):

R¹³R¹⁴C═CR¹⁵R¹⁶ (VI)

R¹⁷R¹⁸C═CR¹⁹R²⁰ (VII)

the olefin (VI) being an exo olefin (terminal double bond) or endo olefin (non-terminal double bond) comprising q carbon atoms; the olefin (VII) comprising r carbon atoms, q is between 4 and 32 and r is between 3 and 40;

thus, in the formulae (VI) and (VII)

R¹⁵ and R¹⁶ represent H and R¹³ and R¹⁴, identical or different, represent an alkyl group, linear or branched, comprising in total, with the carbon atoms carrying the double bond, q carbon atoms; or

R¹³, R¹⁴, R¹⁵ and R¹⁶, identical or different, represent an alkyl group, linear or branched, comprising in total, with the carbon atoms carrying the double bond, q carbon atoms; or

R¹³ represents H and R¹⁴, R¹⁵ and R¹⁶, identical or different, represent an alkyl group, linear or branched, comprising in total, with the carbon atoms carrying the double bond, q carbon atoms;

R¹⁷, R¹⁸, R¹⁹ and R²⁰, identical or different, represent an alkyl group, linear or branched, comprising in total, with the carbon atoms carrying the double bond, q carbon atoms; or

R¹⁷, R¹⁹ and R²⁰ represent H and R¹⁸ represents an alkyl group, linear or branched, comprising a total, with the carbon atoms carrying the double bond, r carbon atoms.

Preferably, for preparing olefins of formula (II) wherein the number of carbon atoms n represents an odd number between 9 and 49 or a number n represents 10, 14, 18, 22, 26, 30, 32, 34, 36, 40, 42, 44, 46, 50:

The lower olefins used in the codimerisation method may for example be of formula (IV) and (V):

R⁵R⁶C═CR⁷R⁸ (IV)

R⁹R¹⁰C═CR¹¹R¹² (V)

the olefin (IV) being an exo olefin (terminal double bond) or endo olefin (non-terminal double bond) comprising 4t carbon atoms, t being an integer number between 1 and 6

thus, in the formulae (IV) and (V)

R⁷ and R⁸ represent H and R⁵ and R⁶, identical or different, represent an alkyl group, linear or branched, comprising 1 to 12 carbon atoms. or

R⁵, R⁶, R⁷ and R⁸, identical or different, represent an alkyl group, linear or branched, comprising 1 to 12 carbon atoms; or

R⁵ represent H and R⁶, R⁷ and R⁸, identical or different, represent an alkyl group, linear or branched, comprising 1 to 12 carbon atoms;

R⁹, R¹⁰, R¹¹ and R¹², identical or different, represent an alkyl group, linear or branched, comprising 1 to 12 carbon atoms; or

R⁹, R¹¹ and R¹² represent H and R¹⁰ represent an alkyl group, linear or branched, comprising 1 to 12 carbon atoms;

the total number of carbon atoms in formula (IV) being m and the total number of carbon atoms in formula (V) being p.

Preferably, for preparing olefins of formula (II) wherein the number of carbon atoms n represents an odd number between 9 and 49 or a number n represents 10, 14, 18, 22, 26, 30, 32, 34, 36, 40, 42, 44, 46, 50:

The metathesis method is implemented between an olefin comprising q carbon atoms and an olefin comprising r carbon atoms, q and r being integer numbers selected so that q+r is greater than n. This is because the metathesis reaction causes the loss of carbon atoms in the final compound (loss of at least two carbon atoms), the number of carbon atoms lost being dependent on the olefins used and in particular on the nature of substituents of the two carbon atoms of the double bond.

The lower olefins used in the metathesis method may for example be of formulae (VI) and (VII):

R¹³R¹⁴C═CR¹⁵R¹⁶ (VI)

R¹⁷R¹⁸C═CR¹⁹R²⁰ (VII)

the olefin (VI) being an exo olefin (terminal double bond) or endo olefin (non-terminal double bond) comprising 46 carbon atoms, t being between 1 and 6

thus, in the formulae (VI) and (VII)

R¹⁵ and R¹⁶ represent H and R¹³ and R¹⁴, identical or different, represent an alkyl group, linear or branched, comprising 1 to 12 carbon atoms; or

R¹³, R¹⁵ and R¹⁶, identical or different, represent an alkyl group, linear or R¹³, branched, comprising 1 to 12 carbon atoms; or

R¹³ represents H and R¹⁴, R¹⁵ and R¹⁶, identical or different, represent an alkyl group, linear or branched, comprising 1 to 12 carbon atoms;

R¹⁷, R¹⁸, R¹⁹ and R²⁰, identical or different, represent an alkyl group, linear or branched, comprising 1 to 12 carbon atoms; or

R¹⁷, R¹⁹ and R²⁰ represent H and R¹⁸ represents an alkyl group, linear or branched, comprising 1 to 12 carbon atoms;

the total number of carbon atoms of the formula (VI) being q and the total number of carbon atoms of the formula (VII) being r.

The codimerisation step can be implemented in the presence of a catalyst selected from Brönsted acids in solution, for example H₂SO₄, H₃PO₄, HF, methanesulfonic acid, triflic acid (CF₃SO₃H); solid Brönsted acids, for example organic resins, clays, zeolites, H₃PO₄ on silica; Lewis acids, for example ZnCl₂, AlCl₃; organometallic compounds, for example Ni complexes, mixtures of complexes of Ni and Al; ionic liquids, for example [BMIm][N(CF₃SO₂)₂]/HN(CF₃SO₂)₂; clays with lamellar structures such as Montmorillonite; organic resins such as amberlysts, sulfonic resins; organometallic compounds such as for example [LNiCH₂R₂₁][AlCl₄] wherein L=PR²², R²¹ represents an alkyl, linear or branched, comprising 9 carbon atoms, and R₂₂ represents a —CH₂—R²¹ group.

Preferably, the quantity of catalyst used in the codimerisation is between 1000 ppm and 10% by weight, preferably between 1000 ppm and 5% by weight, with respect to the weight of olefin.

The codimerisation step is preferably implemented at a temperature of between 30 and 250° C., preferably between 100 and 200° C.

Particularly advantageously, the olefins can be obtained from isobutene. Preferably, said isobutene is obtained from bioresources, in particular as described in the applications WO 2012052427, WO 2017085167 and WO 2018206262, for example from polysaccharides (sugars, starches, celluloses, etc.).

The metathesis step is implemented by reacting the two olefins in the presence of a metathesis catalyst, in particular a catalyst selected from the catalysts known to a person skilled in the art for metathesis, in particular ruthenium catalysts in particular the 2^{nd} generation Grubbs catalysts, for example benzylidene 1,3-bis(2,4,6-trimethylphenyl)-2-imidazolidinylidene dichloro(tricyclohexyl-phosphine) ruthenium or (1,3-dimesitylimidazolidine-2-ylidene)(tricyclohexylphosphine)benzylidene ruthenium dichloride. The quantity of catalyst is preferably between 50 ppm and 5% by weight Ru element, preferably between 200 ppm and 1%, with respect to the weight of olefin. The reaction is preferably implemented at a temperature between 0 and 150° C., for example between 20 and 100° C. The medium next undergoes a purification step, for example the reaction medium is dissolved in a solvent, for example toluene, and then the mixture obtained is filtered, for example on neutral alumina.

The olefins according to the invention can be used for formulating cosmetic compositions, plasticiser compositions or lubricant compositions.

The olefins of the invention may also be hydrogenated to corresponding alkanes or undergo reactions transforming them into functionalised alkanes, said alkanes being able to be used for formulating cosmetic compositions, plasticising compositions or lubricating compositions.

The present application also relates to the use of branched alkanes according to the invention or a mixture of branched alkanes according to the invention for formulating cosmetic compositions, plasticising compositions or lubricating compositions.

The present application will now be described by means of the following examples.

### EXAMPLE 1: DIMERISATION OF ISOOCTENE

The following are loaded into a stirred autoclave, closed and put under inert atmosphere:
100 g of isooctene
10 g of Montmorillonite
5 g of isooctane

Heating is carried out gradually and dimerisation commences at around 150° C.

The temperature is continued to be increased up to 200° C.

The mixture is maintained under stirring and at this level temperature for 3 hours.

The reaction mixture is cooled to ambient temperature.

The Montmorillonite catalyst is separated from the liquid phase by filtration.

The liquid phase is diluted in a cyclohexane solvent for analysis requirements.

The conversion of isooctane is between 70 and 95%. The yields of dimerisation products of isooctane (hexaisododecenes) are between 50 and 90%.

### EXAMPLE 2: DIMERISATION OF ISOOCTENE

The catalyst used in this example is sold by AXEN and is a solution of dichloroalkylaluminium at 50% by weight in a C₆-C₈ paraffinic petroleum fraction, and nickel-based liquid catalyst.

The following are loaded into a stirred autoclave closed and put under inert atmosphere:
100 g of isooctene
0.45 g of the catalytic solution defined above

The reaction mixture is maintained at between 45 and 50° C. for 2 hours.

The mixture is cooled to ambient temperature.

The mixture is treated by a basic aqueous solution of sodium carbonate or of soda and the organic and aqueous phases are next separated by settling.

The organic phase is analysed.

The conversion of the isooctane is between 70 and 100%.

The yields of dimerisation products of the isooctane (hexaisododecenes) are between 60 and 90%.

### EXAMPLE 3: HYDROGENATION OF THE COMPOUNDS RESULTING FROM EXAMPLES 1 AND 2

After 3 purges under nitrogen stream, the following are loaded into a hydrogenation reactor (stirring and maintenance under pressure):
100 g of dimer of the isooctane obtained at examples 1 and 2
5 g of Raney Ni catalyst
50 g of isooctane.

The stirred mixture is heated to a temperature of 80° C.

Hydrogen is introduced while adjusting the pressure to a constant value of 20 atmospheres.

The reaction mixture is kept stirred, at 50° C., under constant pressure of hydrogen for a period of 3 hours.

At the end of the reaction, the excess hydrogen is eliminated by pressure reduction and the top of the reactor is purged 3 times with nitrogen.

The reaction medium is diluted in cyclohexane for analytical purposes.

The reaction medium is analysed:

The conversion of dimer of the isooctane is 100%.

The yield of hydrogenated dimer (branched alkane according to the invention) is 100%.

### EXAMPLE 4: METATHESIS OF ISOOCTANE AND OCTENE]

The following are added successively in a Schlenk flask: 18.5 mmol of isooctene (2.1 mL) 4 mmol of octene (0.45 mL) and (1,3-dimesitylimidazolidine-2-ylidene)(tricyclohexylphosphine)benzylidene ruthenium dichloride (68 mg, 0.08 mmol). The solution is heated to 55° C. and regularly degassed. After 40 h, 0.3 mL of ethyl vinyl ether is added. The product is dissolved in toluene (50 mL) and then filtered on neutral alumina. 42% of product (C₁₄ olefin) is obtained after the solvent is evaporated.

An identical method can be implemented for the following reactions:

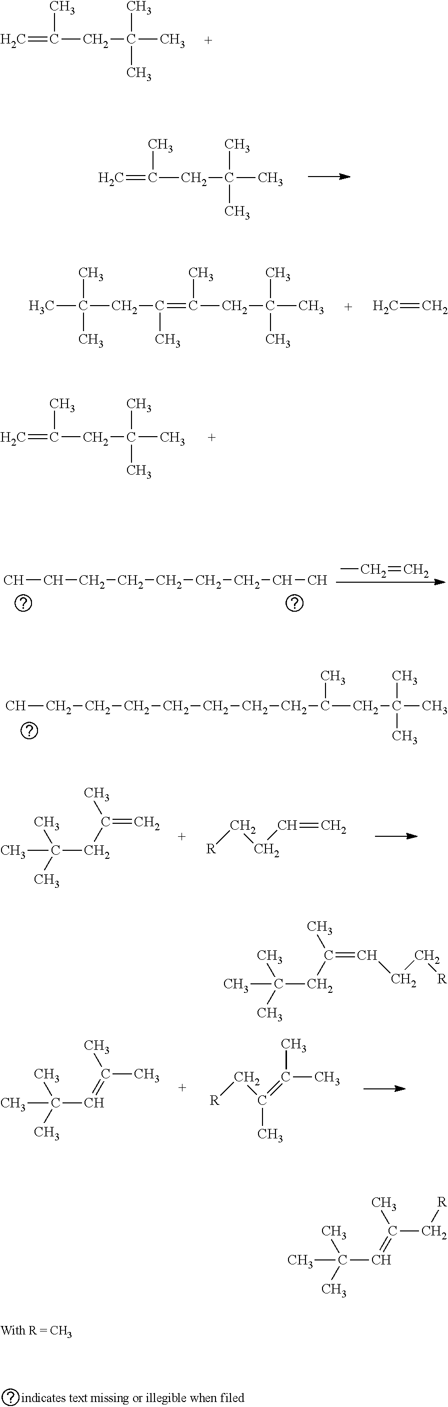

### EXAMPLE 5: CODIMERISATION

The following are loaded into a stirred autoclave, closed and put under inert atmosphere:
100 g of isooctene
100 g of n-octene
10 g of Montmorillonite
5 g of isooctane

Heating is carried out gradually and codimerisation commences at around 150° C.

The temperature is continued to be increased up to 200° C.

The mixture is maintained under stirring and at this level temperature for 3 hours.

The reaction mixture is cooled to ambient temperature.

The Montmorillonite catalyst is separated from the liquid phase by filtration. The liquid phase is diluted in a cyclohexane solvent for the requirements of analysis.

The conversion is between 70 and 95%. The yields of hexadodecene and products of the codimerisation of isooctane with n octene are between 50 and 90%.

An identical method can be implemented for the following reactions:

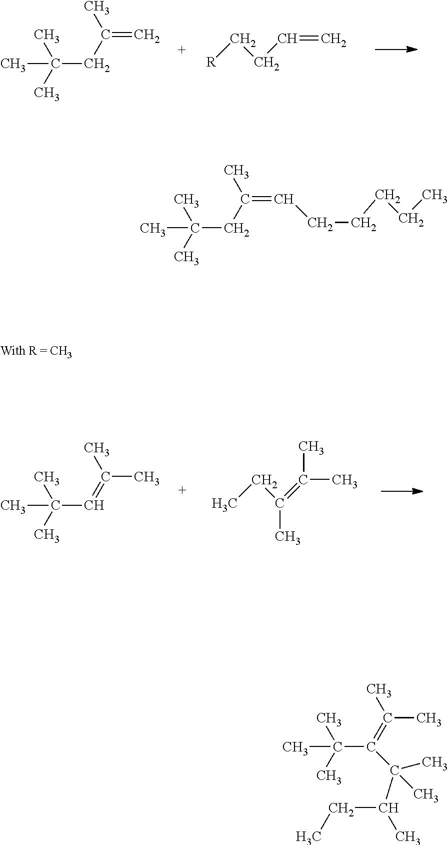

### EXAMPLE 6: OBTAINING C₁₂ OLEFINS

The methods of the invention (codimerisation and metathesis) can be implemented to obtain olefins comprising 12 carbon atoms, in accordance for example with the following reaction diagrams:

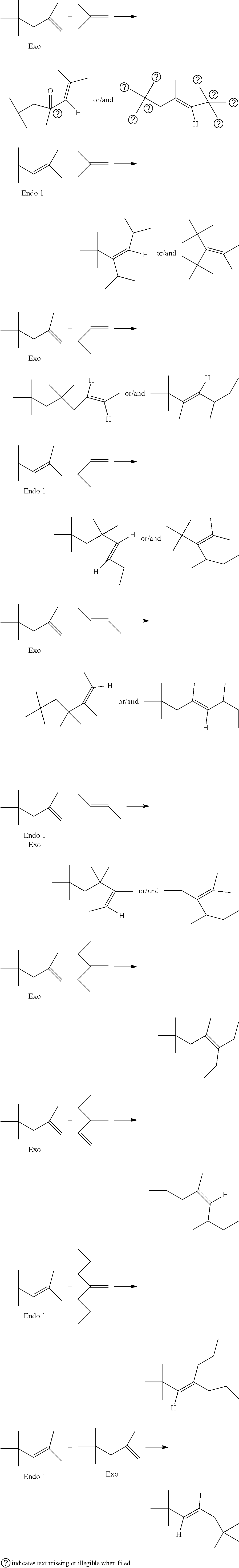

1. A branched alkane of the following formula (I): 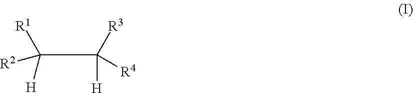
R¹, R², R³ and R⁴, identical or different, are selected from H, alkyls, linear or branched, comprising 1 to 46 carbon atoms and the total number of carbon atoms in all the R¹, R², R³ and R⁴ groups being between 7 and 48;
provided that:
at most two of the R¹, R², R³ and R⁴ groups are H
one of the R¹, R², R³ or R⁴ groups includes or is a tert-butyl group.

2. The branched alkane according to claim 1, with one of R¹, R², R³ or R⁴ representing a methyl group.

3. A mixture comprising at least two branched alkanes according to claim 1, for which the n carbon atoms are identical or different.

4. The mixture according to claim 3, free from aromatic compounds.

5. A branched olefin of formula (III) 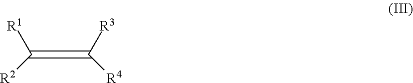
R¹, R², R³ and R⁴, identical or different, are selected from H, alkyls, linear or branched, comprising 1 to 46 carbon atoms and the total number of carbon atoms in all the R¹, R², R³ and R⁴ groups being between 7 and 48;
provided that:
at most two of the R¹, R², R³ and R⁴ groups are H
one of the R², R², R³ or R⁴ groups includes or is a tert-butyl group.

6. A mixture comprising at least two branched olefins according to claim 5, the n carbon atoms are identical or different.

7. A method for obtaining a branched alkane according to claim 1 or of a mixture comprising at least two of the branched alkanes, comprising a step of dehydrogenation of a branched olefin of formula (III) 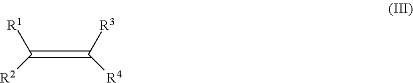
R¹, R², R³ and R⁴, identical or different, are selected from H, alkyls, linear or branched, comprising 1 to 46 carbon atoms and the total number of carbon atoms in all the R¹, R², R³ and R⁴ groups being between 7 and 48;
provided that:
at most two of the R¹, R², R³ and R⁴ groups are H
one of the R¹, R², R³ or R⁴ groups includes or is a tert-butyl group or a mixture comprising at least two of the branched olefins.

8. The method according to claim 7, wherein the branched olefin or the mixture is obtained by dimerisation of a mixture of branched olefin isomers comprising n/2 carbon atoms when n represents 16, 24, 32, 40 or 48, by codimerisation or by metathesis of lower olefins.

9. The method according to claim 8, wherein the branched olefin mixture comprising n/2 carbon atoms is obtained from bioresources.

10. The method according to claim 8, wherein the dimerisation step is implemented in the presence of a catalyst selected from Brönsted acids in solution; solid Brönsted acids; Lewis acids; organometallic compounds; ionic liquids; clays with lamellar structures; organometallic compounds.

11. The method according to claim 8, wherein the codimerisation method is implemented with lower olefins of formulae (IV) and (V):
R⁵R⁶C═CR⁷R⁸ (IV)
R⁹R¹⁰C═CR¹¹R¹² (V)
the olefin (IV) being an exo olefin (terminal double bond) or endo olefin (non-terminal double bond) comprising m carbon atoms, the olefin (V) comprising p carbon atoms, with m+p=n with n representing an integer between 9 and 50, m between 4 and 32 and p between 3 and 46,
thus, in the formulae (IV) and (V)
R⁷ and R⁸ represent H and R⁵ and R⁶, identical or different, represent an alkyl group, linear or branched, comprising in total, with the carbon atoms carrying the double bond, m carbon atoms; or
R⁵, R⁶, R⁷ and R⁸, identical or different, represent a linear or branched alkyl group comprising in total, with the carbon atoms carrying the double bond, m carbon atoms; or
R⁵ represents H and R⁶, R⁷ and R⁸, identical or different, represent a linear or branched alkyl group comprising in total, with the carbon atoms carrying the double bond, m carbon atoms;
R⁹, R¹⁰, R¹¹ and R¹², identical or different, represent an alkyl group, linear or branched, comprising in total, with the carbon atoms carrying the double bond, p carbon atoms; or
R⁹, R¹¹ and R¹² represent H and R¹⁰ represents an alkyl group, linear or branched, comprising in total, with the carbon atoms carrying the double bond, p carbon atoms.

12. The method according to claim 8, wherein the metathesis method is implemented with lower olefins of formulae (VI) and (VII):
R¹³R¹⁴C═CR¹⁵R¹⁶ (VI)
R¹⁷R¹⁸C═CR¹⁹R²⁰ (VII)
the olefin (VI) being an exo olefin (terminal double bond) or endo olefin (non-terminal double bond) comprising q carbon atoms; the olefin (VII) comprising r carbon atoms, q is between 4 and 32 and r is between 3 and 40;
thus, in the formulae (VI) and (VII)
R¹⁵ and R¹⁶ represent H and R¹³ and R¹⁴, identical or different, represent an alkyl group, linear or branched, comprising in total, with the carbon atoms carrying the double bond, q carbon atoms; or
R¹³, R¹⁴, R¹⁵ and R¹⁶, identical or different, represent an alkyl group, linear or branched, comprising in total, with the carbon atoms carrying the double bond, q carbon atoms; or
R¹³ represents H and R¹⁴, R¹⁵ and R¹⁶, identical or different, represent an alkyl group, linear or branched, comprising in total, with the carbon atoms carrying the double bond, q carbon atoms;
R¹⁷, R¹⁸, R¹⁹ and R²⁰, identical or different, represent an alkyl group, linear or branched, comprising in total, with the carbon atoms carrying the double bond, q carbon atoms; or
R¹⁷, R¹⁹ and R²⁰ represent H and R¹⁸ represents an alkyl group, linear or branched, comprising a total, with the carbon atoms carrying the double bond, r carbon atoms.

13. The method according to claim 9, wherein the codimerisation step is implemented in the presence of a catalyst selected from Brönsted acids in solution; solid Brönsted acids; Lewis acids; ionic liquids; clays with lamellar structures; organometallic compounds.

14. The method according to claim 10, wherein the metathesis step is implemented by reacting the two olefins in the presence of a metathesis catalyst.

15. A method for formulating a cosmetic composition, a plasticising composition or a lubricating composition comprising including an alkane according to claim 1 or mixture comprising at least two of the branched alkanes.

16. A mixture comprising at least two branched alkanes according to claim 2, for which the n carbon atoms are identical or different.

17. The mixture according to claim 16, free from aromatic compounds.

18. A method for obtaining a branched alkane according to claim 2 or of a mixture comprising at least two of the branched alkanes, comprising a step of dehydrogenation of a branched olefin of formula (III) 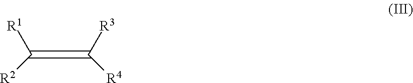
R¹, R², R³ and R⁴, identical or different, are selected from H, alkyls, linear or branched, comprising 1 to 46 carbon atoms and the total number of carbon atoms in all the R¹, R², R³ and R⁴ groups being between 7 and 48;
provided that:
at most two of the R¹, R², R³ and R⁴ groups are H
one of the R¹, R², R³ or R⁴ groups includes or is a tert-butyl group or a mixture comprising at least two of the branched olefins.

19. The method according to claim 9, wherein the dimerisation step is implemented in the presence of a catalyst selected from Brönsted acids in solution; solid Brönsted acids; Lewis acids; organometallic compounds; ionic liquids; clays with lamellar structures; and organometallic compound.

20. The method according to claim 10, wherein the metathesis step is implemented by reacting the two olefins in the presence of a metathesis catalyst selected from the 2^{nd} generation Grubbs catalyst.
